(19) United States
(12) Patent Application Publication
(10) Pub. No.: US 2005/0222275 A1
(43) Pub. Date: Oct. 6, 2005

(54) Use of dmso in the treatment of neurodegenerative diseases caused by prions

(76) Inventors: Ruth Gabizon, Jerusalem (IL); Gideon M. Shaked, Jerusalem (IL)

Correspondence Address: HODGSON RUSS LLP, ONE M & T PLAZA, SUITE 2000, BUFFALO, NY, 14203-2391, US

(21) Appl. No.: 10/486,941

(22) PCT Filed: Aug. 14, 2002
(86) PCT No.: PCT/IL02/00672

§ 371 Date: Dec. 15, 2004

(30) Foreign Application Priority Data
Aug. 16, 2001 (IL) 144949

(51) Int. Cl. A61K 31/10

(52) U.S. Cl. 514/708

(57) ABSTRACT

The present invention relates to a pharmaceutical composition for the treatment and/or delaying the onset of clinical symptoms and/or delaying the progress of a neurodegenerative disorder caused by prions. The composition of the invention comprises as an active ingredient DMSO, and optionally further comprising pharmaceutically acceptable additives and/or diluents. The composition of the invention is particularely applicable in the treatment of neurodegenerative disorders associated with enhanced accumulation of PrP^{Sc} into amyloid plaques. The invention further relates to methods of treatment of a neurodegenerative disorder caused by prions and to the use of DMSO in the preparation of a pharmaceutical composition for the treatment and/or delay of the onset of clinical symptoms and/or delay of progress of neurodegenerative disorders caused by prions.

### FIELD OF THE INVENTION

The present invention relates to the treatment of neurodegenerative disorders caused by prions, like Creutzfeldt-Jakob disease (CJD), with the organic solvent dimethyl sulfoxide (DMSO).

### BACKGROUND OF THE INVENTION

All publications mentioned throughout this application are fully incorporated herein by reference, including all references cited therein.

Prion diseases, also known as TSEs (transmissible spongiform encephalopathies), are a group of fatal neurodegenerative diseases of animals and humans recognized neuropathologically by the classic triad of spongy degeneration (affecting any part of the cerebral grey matter), neuronal loss and the proliferation and hypertrophy of astrocytes [Beck, E. & Daniel, P. M. Postgrad Med J 45(524), 361-70 (1969)]. The disease originates from the conversion of PrP^{C} molecules into protease-resistant and insoluble PrP^{Sc} molecules by a yet not deciphered mechanism in which PrP^{Sc} serves as a template. The hallmark of these diseases is the accumulation of the abnormal isoform of prion protein (PrP^{Sc}) in the brain, forming amyloid plaques [Prusiner, S. B. & DeArmond, S. J. Lab Invest 56(4), 349-63 (1987)]. Among the animal diseases, the most prevalent is the bovine spongiform encephalopathy (BSE), commonly known as “mad cow” disease.

The sporadic form of Creutzfeldt-Jakob disease (CJD) accounts for approximately 85 percent of all cases of prion disease in humans, whereas infectious and inherited prion diseases account for the rest. The common symptoms of CJD are dementia, followed by loss of coordination, although the sequence of these events can be reversed. The inherited form of CJD affects 10-15% of the patients, and three variants have been characterized. Very rarely, the disease can be acquired through infection, as an inadvertent consequence of a medical procedure. In the sporadic cases, the mean age of onset is 60 years old, and the disease lasts for about 8 months. For the inherited disease, these parameters vary according to the allelic variant, and the mean age of onset is between 45 to 55 years old, while it may last from 8 to 50 months.

In Europe, the increasing number of patients with a new variant of CJD, called variant CJD (vCJD) has been associated with the consumption of prion-tainted beef products [Will, R. G. et al. Lancet 347, 921-5 (1996)]. The current understanding is that vCJD is contracted by horizontal transmission through beef or lamb products derived from animals contaminated with BSE or scrapie, respectively.

Nowadays, CJD constitutes a major health risk, accounting for approximately 85 percent of all cases of human prion disease. The incidence of Creutzfeldt-Jakob disease is approximately one case per 1 million people, but among persons between the ages of 60 and 74 years, the incidence is nearly 5 per 1 million [Prusiner, S. N Engl J Med 344(20), 1516-1526 (2001)].

Other prion diseases in humans include Kuru, Gerstmann-Sträussler-Scheinker syndrome (GSS syndrome) and fatal familial insomnia (FFI). In addition, more than 20 pathogenic mutations in the PrP gene have already been documented [Prusiner, S. B. Philos Trans R Soc Lond B Biol Sci 343, 447-63 (1994)].

PrP^{Sc} is the only identified component of the prion, the proteinaceous agent causing prion disease [Prusiner, S. B. et al. Cell 93(3), 337-48 (1998)]. PrP^{Sc} is a conformational aberrant isoform of PrP^{C}, a widely distributed cell-surface glycoprotein of unknown function. The difference between the two PrP isoforms seems to be conformational rather than chemical. PrP^{C} conformation is mostly α-helix, while PrP^{Sc} contains a considerable amount of β-sheet (FIG. 1). However, both proteins share the same amino acid sequence and have the same apparent MW of 33-35 Kd. The level of PrP^{C} expression seems to play a central role in the accumulation of PrP^{Sc}, since PrP^{C} is the source for PrP^{Sc} production. This has been shown both in tissue culture and in transgenic mice. The conversion of α-helical PrP^{C} molecules into β-sheet enriched PrP^{Sc} has been postulated as the cause for the propagation of prion diseases. The pathway for PrP^{Sc} synthesis either may feature the formation of PrP^{C}-PrP^{Sc} heterodimers only, or may include the formation of seeds of aggregation comprised of mediator molecules as well. Currently, the mechanisms involved in this process remain unclear.

The organic solvent dimethyl sulfoxide (DMSO) has demonstrated both in vitro and in vivo effects on amyloidotic diseases (diseases where there is amyloid plaque formation, like in prion diseases). In vitro data, from experiments concerned with the degradation of amyloid proteins, suggest that DMSO blocks the assembly of amyloid fibrils but does not interfere with the disassembly of preexisting fibrils [Kito, S. et al. Ann N Y Acad Sci 411, 52-66 (1983)]. Moreover, DMSO has been shown to block the formation of an Aβ peptide intermediate with a high β-sheet content, which is a controlling step in the process of self assembly into amyloid plaques during Alzheimer's disease [Shen, C. L. & Murphy, R. M. Biophys J 69, 640-651 (1995)].

DMSO treatment of scrapie-infected neuroblastoma cells (ScN2a) interfered with the formation of PrP^{Sc} from newly synthesized PrP^{C}. This result suggested that DMSO could act as a “chemical chaperone” to stabilize the α-helical conformation of PrP^{C} and prevent it from undergoing a conformational change that would produce PrP^{Sc} [Tatzelt, J., Prusiner, S. B. & Welch, W. J EMBO Journal 15, 6363-6373 (1996)]. In contrast, the inventors have shown that DMSO does not affect the conversion process as demonstrated by Tatzelt, but rather it affects the accumulation process from PrP^{Sc} into amyloid plaques, which are sedimented in the brain in a state of disease. Interestingly, treatment of brain homogenates with glycerol, also suggested by Tatzelt et al. as a “chemical chaperone”, showed no detectable effect (Inventor's unpublished results).

A murine model for caseic amyloidosis has shown that oral administration of DMSO not only prevented the development of the disease, but also seemed to cause its regression in some cases [Murav'ev, I. O. reumatologia 1, 44-46 (1990)].

DMSO has been available for medical study since 1963. It has FDA approval only for the symptomatic treatment of interstitial cystitis, but has been shown beneficial for the treatment of leukemia and cancer, cerebral ischaemia, head trauma and infection [Regelson, W. & Harkins, S. W. Ann N Y Acad Sci 826, 348-74. (1997)]. When given as a continuous treatment to humans, DMSO has no serious side effects [Ravid. M, S. J., Lang. R. Ann Rheum Dis 41, 587-592 (1982); Brobyn, R. D. Ann NY Acad Sci 243, 497-505 (1975)].

There are a few reports on the clinical use of DMSO in amyloidotic diseases. Following a single dose of DMSO, urine of patients suffering from amyloidotic diseases contains material that can form fibrils that can be stained with Congo Red. This result suggests that DMSO may cause the breakdown of amyloid fibrils into a conformation which is transported from body tissues and eliminated through the kidneys [Ravid, M., Kedar, I. & Sohar, E. Lancet 1, 730-731 (1977)]. In addition, following chronic administration of DMSO to humans suffering from Primary and Familial Amyloidosis (FAP), about 50% of the patients showed clinical improvement. However, the mechanism of action of DMSO is still not clearly understood.

In search for a therapeutic treatment of CJD and other prion diseases, the inventors have found that treatment with DMSO improves the condition of the patient. It is therefore an object of the present invention to use DMSO and pharmaceutical compositions containing DMSO in the treatment of prion diseases, particularly in humans and specifically CJDs. This and other objects of the invention will be elaborated on the description proceeds.

### SUMMARY OF THE INVENTION

The object of this invention relates, in one aspect, to a pharmaceutical composition and in a second aspect, to a method for the treatment and/or delay of the onset of clinical symptoms and/or delay of the progress of a neurodegenerative disorder caused by prions, comprising as active ingredient DMSO, and optionally further comprising pharmaceutically acceptable additives and/or diluents.

Specific neurodegenerative disorders are those associated with enhanced accumulation of PrP^{Sc} into amyloid plaques, and particularly disorders associated with excess of PrP^{Sc} in the brain. These neurodegenerative disorders are progressive neurodegenerative disorders, exemplified by Creutzfeldt-Jacob disease (CJD), variant CJD (vCJD), Kuru, Gerstmann-Sträussler-Scheinker syndrome (GSS syndrome) and fatal familial insomnia (FFI), specifically a Creutzfeldt-Jakob disease.

The pharmaceutical composition is preferably for oral administration, from one to six times per day, preferably two to four times per day, and most preferably three times per day, and it is in the form of an aqueous solution of DMSO, wherein the concentration of DMSO is from about 1% (w/v) to about 100% (w/v), preferably 20% (w/v), comprising a dosage of DMSO of about 0.2 g/kg daily, or from about 0.1 g to about 30 g daily, preferably 5 g daily.

The invention relates to a method for treating and/or delaying the onset of clinical symptoms and/or delaying the progress of a prion disease, comprising administering to a CJD patient a therapeutically effective amount of DMSO or of a pharmaceutical composition comprising as active ingredient DMSO and optionally further comprising pharmaceutically acceptable additives and/or diluents.

The method of the invention is particularly intended for the treatment of neurodegenerative disorders caused by prions, that are associated with enhanced accumulation of PrP^{Sc} into amyloid plaques and with excess of PrP^{Sc} in the brain, such as Creutzfeldt-Jacob disease, vCJD, Kuru, GSS syndrome and FFI, specifically a Creutzfeldt-Jakob disease.

In another aspect, the invention comprises the use of DMSO in the treatment and/or delay of the onset of clinical symptoms and/or delay of the progress of a neurodegenerative disorder caused by prions, such as CJD, vCJD, Kuru, GSS syndrome and FFI, specifically Creutzfeldt-Jakob disease.

A further aspect of this invention is the use of DMSO in the preparation of a pharmaceutical composition for the treatment and/or delay of the onset of clinical symptoms and/or delay of progress of neurodegenerative disorders caused by prions, that are associated with enhanced accumulation of PrP^{Sc} into amyloid plaques and with excess of PrP^{Sc} in the brain, and are manifested as any one of the progressive neurodegenerative disorders exemplified by Creutzfeldt-Jacob disease, vCJD, Kuru, GSS syndrome and FFI, specifically a Creutzfeldt-Jakob disease.

The invention will be described in more detail on hand of the following drawings.

### BRIEF DESCRIPTION OF THE DRAWINGS

FIG. 1a-b: Schematic of PrP^{C} and PrP^{Sc}.

FIG. 1(a) Ribbon drawing of the NMR of recombinant Syrian Hamster PrP (residues 90 to 231).

FIG. 1(b) Model of the estimated structure of PrP^{Sc}, illustrating the dramatic increase in the β-sheet structure.

FIG. 2: Oral DMSO treatment can prolong life span of Scrapie (IC) infected Hamsters.

Infectivity rates are depicted on a logarithmic scale. The higher values indicate acute infectivity rates. Abbreviations: Inocu. (inoculation), cont. (control).

FIG. 3: Oral DMSO treatment can prolong life span of Scrapie (IP) infected Hamsters.

Infectivity levels are depicted on a linear scale (dpi). Higher values indicate increased animal life span, hence lower infectivity rates. Abbreviations: Infec. (infectivity), cont. (control), treat. (treatment).

FIG. 4a-b: Lower PrP^{Sc} levels in DMSO-treated scrapie-infected hamsters.

Brain homogenates from scrapie hamsters from different DMSO treatment groups were Western blotted following Proteinase K treatment. PrP^{Sc} concentration was estimated according to the intensity of the bands.

FIG. 4(a) Lane 1, sample from untreated animals harvested at 90 dpi; lanes 2 and 3 represent 10 and 100 times dilution of the sample from lane 1, respectively.

FIG. 4(b) Lane 1, sample from DMSO-treated animals harvested at 110 dpi; lane 2, sample from a DMSO-treated animal harvested at 90 dpi; lanes 3 and 4, sample from lane 2 diluted 10 and 100 times, respectively.

The sample from the untreated animal at 90 dpi (a, first lane) shows similar intensity to the sample from a DMSO-treated animal at 110 dpi (b, first lane). At 90 dpi, the sample from an untreated animal displayed about 10 times more PrP^{Sc} than the sample from a DMSO-treated animal (compare lane 2 in FIGS. 4a and 4b).

FIG. 5a-b: PrP levels in untreated animals at 60 dpi are more similar to PrP levels in DMSO-treated animals at 110 dpi.

Western blots prepared from hamster brain homogenates, comparing between:

FIG. 5(a) sample from an untreated animal at 60 dpi undiluted, 10 and 100 times diluted (lanes 1, 2 and 3, respectively).

FIG. 5(b) three time points of DMSO-treated animals (60, 90 and 110 dpi, lanes 1, 2, and 3, respectively).

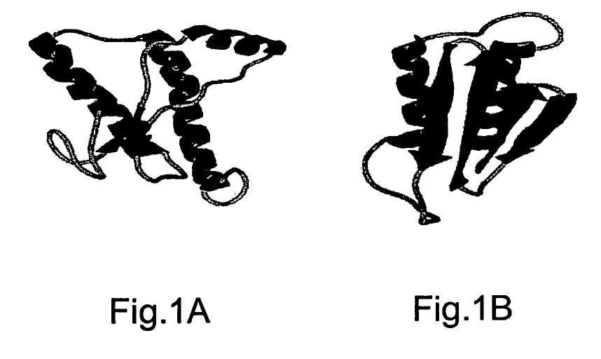

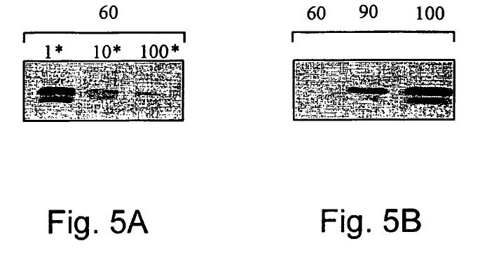

A difference of about 100 fold is evident among the samples from 60 dpi (compare lane 1 in FIGS. 5a and 5b). A similar PrP concentration is present in the sample from untreated animals at 60 dpi and the sample from DMSO-treated animals at 110 dpi (compare lane 1 from FIG. 5a with lane 3 from FIG. 5b).

### DETAILED DESCRIPTION OF THE INVENTION

For the purposes of clarity, the following terms are defined herein:
CJD: Creutzfeldt-Jakob disease. A progressive neurodegenerative disorder that occurs in humans caused by abnormal accumulation of the prion protein (PrP), in which the common symptoms are dementia and loss of coordination.
vCJD: variant CJD. A recently described form of CJD contracted, most likely, through the consumption of BSE or scrapie-contaminated beef or lamb derived products.
DMSO: dimethyl sulfoxide.
Neurodegenerative disorder: A disorder or condition that results from degeneration of the central nervous system, due to abnormalities in the processing of neuronal proteins. Defective processing leads to the accumulation of one or more specific proteins, causing various diseases. The most common neurodegenerative disorder is Alzheimer's disease. Also known are prion diseases, Parkinson's disease, amyotrophic lateral sclerosis, frontotemporal dementia, Huntington's disease, and spinocerebellar ataxia.
Prion disease: A fatal neurodegenerative disorder caused by the deleterious accumulation of the abnormal form of prion protein. Prion diseases have a broad spectrum of clinical manifestations, including dementia, ataxia, insomnia, paraplegia, paresthesias, and deviant behavior. Neuropathological findings range from an absence of atrophy to widespread atrophy, from minimal to widespread neuronal loss, from sparse to widespread vacuolation or spongiform changes, from mild to severe reactive astrocytic gliosis, and from an absence of PrP amyloid plaques to an abundance of plaques. None of these findings except the presence of PrP amyloid plaques is unequivocally diagnostic of a prion disease.
Prion protein (PrP): Infectious protein that behaves like a pathogen. Prions are capable of reproducing by recruiting normal cellular prion protein (PrP^{C}) and stimulating its conversion to the disease-causing isoform (PrP^{Sc}).
PrP^{C}: An GPI-anchored glycoprotein of unknown function, which constitutes the normal, non-infectious form of the prion protein. Upon its conversion into PrP^{Sc}, it becomes pathogenic. NMR studies have shown that the tertiary structure of PrP^{C} is rich in α-helices.
PrP^{Sc}: A protease-resistant and insoluble form of prion, originated by the conversion of Prp^{C} into PrP^{Sc}. The accumulation of PrP^{Sc} in the brain forms amyloid plaques, which are the hallmark of prion disease. A model of the tertiary structure of PrP^{Sc} postulates a structure rich in β sheets.

Prions cause neurodegenerative disorders in mammals throughout the world, the best known being Creutzfeldt-Jakob disease in humans, bovine spongiform encephalopathy (BSE), and scrapie in sheep. Although significant advances have been made in understanding the molecular and cellular basis of prion diseases, these efforts have not yet led to promising therapeutic interventions. The emergence of more than 50 cases of a new variant CJD in humans (vCJD), that seems to be caused by BSE prions from cattle, has heightened the urgency for the development of effective therapeutics.

As a first aspect, the present invention relates to a pharmaceutical composition comprising as active ingredient the organic solvent DMSO, having therapeutic properties for the treatment of neurodegenerative disorders caused by prions, and a pharmaceutically acceptable diluent.

In a specifically preferred embodiment of the present aspect, the pharmaceutical composition of the invention comprises the organic solvent DMSO, having therapeutic properties for the treatment of neurodegenerative disorders associated with enhanced accumulation of PrP^{Sc} into amyloid plaques, and a pharmaceutically acceptable diluent.

In another embodiment, the pharmaceutical composition of the invention comprises the organic solvent DMSO, having therapeutic properties for the treatment of neurodegenerative disorders associated with excess PrP^{Sc} in the brain, and a pharmaceutically acceptable diluent.

In yet another embodiment, the pharmaceutical composition of the invention comprises the organic solvent DMSO, having therapeutic properties for the treatment of neurodegenerative disorders, such as disorders associated with a Creutzfeldt-Jakob disease, and a pharmaceutically acceptable diluent. The composition of the invention may also be effective in treating other prion diseases in humans, including Kuru, Gerstmann-Sträussler-Scheinker syndrome (GSS syndrome) and/or fatal familial insomnia (FFI).

The pharmaceutical composition is for one to six times per day use, preferably two to four times per day use, and most preferably three times per day use by a subject in need. Hucker et al. have shown that DMSO reaches its peak concentration 4 hours following oral administration, and have suggested six doses a day for maintenance of maximal blood level [Hucker, H. B. et al. J Pharmacol Ther 155, 309-317 (1967)].

In a further embodiment of the invention, the pharmaceutical composition comprises a dosage of active ingredient of about 0.1 g to about 30 g, preferably about 1 g to 15 g, most preferably about 2.5 g to about 7.5 g and specifically 5.0 g per dosage. The concentration of DMSO in the pharmaceutical composition is from about 1% (w/v) to about 100% (w/v), preferably from about 5% to about 50% (w/v), and most preferably 20% (w/v).

A preferred diluent is water, but other aqueous diluents, for example fruit juice, are also contemplated by the invention.

Likely additives to the pharmaceutical composition comprise urea and ethanol, in concentrations that are not harmful to the recipient. Urea and ethanol have the beneficial property of reducing the unwanted malodorous breath and foul taste that result from ingestion of DMSO.

Acceptable carriers, excipients, or stabilizers are non-toxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol and sorbitol; slat-forming counter-ions such a sodium; and/or non-ionic surfactants such as Tween, Pluronics™ or polyethylene glycol (PEG).

The pharmaceutical compositions of the invention can be prepared in dosage units forms. The dosage unit forms can be capsules containing DMSO. In such case the capsules should be made of a material that is not affected by DMSO. The dosage forms may also include sustained release devices. The compositions may be prepared by any of the methods well known in the art of pharmacy. Such dosage forms encompass physiologically acceptable carriers that are inherently non-toxic and non-therapeutic. Examples of such carriers include ion exchangers, alumina, aluminum stearate, lecitin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of DMSO include polysaccharides such as sodium carboxymethylcellulose or methylcellulose, polyvinylpyrrolidone, polyacrylasts, polyoxyethylene-block polymers and PEG. For all administrations, conventional delivery forms are suitably used. Such forms include for example, microcapsules, nano-capsules, liposomes, plasters, inhalation forms, nose sprays, sublingual tablets, and sustained-release preparations.

The preparation of pharmaceutical compositions is well known in the art and has been described in many articles and textbooks, see e.g., Remington's Pharmaceutical Sciences, Gennaro A. R. ed., Mack Publishing Company, Easton, Pa., 1990, and especially pages 1521-1712 therein, fully incorporated herein by reference.

As a second aspect, the present invention relates to the therapeutic method of treatment with DMSO or with the above-described pharmaceutical composition.

The magnitude of a therapeutic dose of DMSO will of course vary with the group of patients (age, sex, etc.), with the stage of the disease, and with the route of administration. In any case, the attending physician will determine the therapeutic dose.

Any suitable route of administration may be employed for providing a mammal, especially a human, with an effective dosage of DMSO or the pharmaceutical composition. Oral administration may be preferred, but treatment may be adapted to other routes of administration, for example by topical application or invasive administration techniques. The term “invasive” as used herein is to be taken to mean intra-cerebral, intraspinal, intramuscular, intravenous, intraperitoneal, subcutaneous, intra-articular, intrasynovial or intrathecal administration.

An “effective amount” of the DMSO or the composition of the invention to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer DMSO until a dosage is reached that achieves the desired effect. A typical daily dosage for systemic treatment might range from about 3 g up to 30 g or more, depending on the factors mentioned above, wherein the concentration of DMSO is from about 1% (w/v) to about 100% (w/v).

“Treatment” refers to therapeutic treatment. Those in need of treatment include those already with the disease or disorder, whether at clinical or pre-clinical stage.

“Mammal” for purposes of treatment refers to any animal classified as a mammal including, human, domestic and farm animals, and zoo, sports, or pet animals, such as dogs, horses, cats, cows, etc. Preferably, the mammal is human.

In a specific embodiment, the mammal treated by the method of the invention is suffering from dementia or lack of coordination, which is the result of a neurodegenerative disease caused by prions.

Another aspect of this invention is the use of DMSO in the treatment of neurodegenerative disorders caused by prions, specifically a Creutzfeldt-Jakob disease.

In another embodiment, the present invention is the use of DMSO in the preparation of a pharmaceutical composition for the treatment of neurodegenerative disorders associated with enhanced accumulation of PrP^{Sc} into amyloid plaques, particularly neurodegenerative disorders associated with excess PrP^{Sc} in the brain.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Brain Homogenates

Brains were homogenized in 10 volumes of homogenizing buffer (10 mM Tris-HCl, pH 7.5, 300 mM Sucrose). Following centrifugation (2000 rpm, 15 min, 4° C.), the supernatant was recovered and frozen (−80° C.). For the production of cell membranes from these brain homogenates, the supernatant was diluted 1:40 with homogenizing buffer and subsequently centrifuged at 20000 rpm for 30 min at 4° C. The pellet was resuspended in 40 ml of hypotonic solution (10 mM Tris-HCl, pH 7.5), and centrifuged again (20000 rpm, 30 min, 4° C.). The pellet from the last centrifugation, consisting only of cell membranes, was resuspended in hypotonic buffer and frozen (−80° C.).

Inoculations and Scrapie Diagnosis

Hamsters were inoculated intracerebrally or intraperitoneally with 50 μl of the samples (5 μl of brain cell membranes diluted 1:10 in 1% BSA/PBS solution). Following inoculation, animals were examined daily for Scrapie-associated symptoms. Infectivity titers were calculated from incubation time (days) according to Prusiner et al. [Prusiner, S. et al. Biochemistry 19(21), 4883-4891 (1980)]. At certain assays compounds were administered via drinking water as described. The water bottles for DMSO-treated animals were glass bottles, light protected and capped with cork because this solvent can rapidly dissolve rubber [O'Dwyer, P. J. et al. Cancer 62(5), 944-948 (1988)]. Water was replaced daily. Animals were monitored for clinical signs of the disease on a daily basis.

Western Blot

Western Blots were carried out in the classical method described in ‘Molecular Cloning—A laboratory manual by Sambrock et al., Cold Spring Harbor Laboratory Press, 2^{nd} edition, 1989’.

Protocol for Patient Treatment

DMSO was dissolved in double-distilled water (20 g/100 ml). 25 ml of this solution was ingested by the patient three times per day. The DMSO solution was stored in an airtight light-protected container. Care was taken that the solution was not exposed to plastic. The solution was well shaken before use.

The liver, renal and ophthalmic functions of the patient should be monitored every six months.

### Example 1

### Syrian Hamsters as a Model for Scrapie Infection

The experimental model for prion disease in rodents is a prototype of several fatal spongiform neurodegenerative diseases of humans and animals [Prusiner, S. B. et al. Ann Neurol 11(4), 353-8 (1982)]. Therefore, the scrapie-infected Hamster model was used to test the effect of DMSO in the infectious rate of the disease and in the level of PrP^{Sc} in the brain.

DMSO was administrated to intra-cerebral (IC) or intra-peritoneal (IP) scrapie-infected Syrian hamsters. Treatment with 7.5% (w/v) DMSO dissolved in drinking water was performed for different lengths of time. Water was provided ad libitum.

Scrapie Infectivity in IC-Inoculated Animals Treated with DMSO

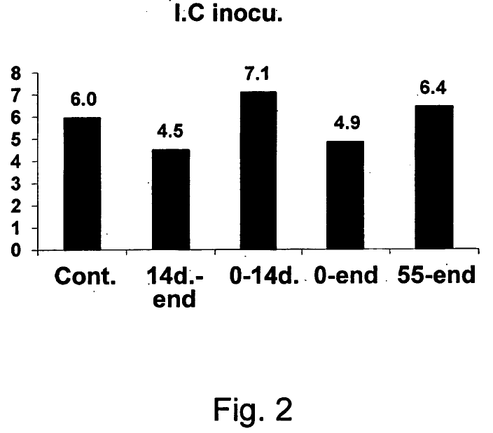

IC-infected animals were divided in five groups. Each group received DMSO according to one of the following treatment plans: control (no DMSO), from 0 dpi (days post-infection) to disease (O-end); from 0 to 14 dpi (0-14); from 14 dpi to disease (14-end); or from 55 dpi to disease (55-end). The results are described in Table 1 and in FIG. 2.

TABLE 1
Oral DMSO treatment can prolong life span of IC scrapie-infected Hamsters
Summary of the results of infectious rates in DMSO-treated animals under
different treatment plans comparing to control untreated animals. Infectious
rates are indicated according to a logarithmic scale [Prusiner, S.B. et al. (1982),
ibid.]. The higher values indicate acute infectious rates.

| | | | | | | Variance |
|---|---|---|---|---|---|---|
| DMSO | Number | Incubation | | | Percentage | from control |
| treatment | of | period | Standard | Infectivity | of infectivity | group |
| (dpi) | animals | (days)^{a} | error | (Log I.D 50) | (%)^{b} | [P < 0.05 ] |
| control | 20 | 93 | 1.36 | 6.0 | 100 | |
| 0-end | 10 | 101 | 3.67 | 4.9 | 3.16 | + |
| 14-end | 10 | 107 | 1.57 | 4.5 | 3.16 | + |
| 0-14 | 5 | 85 | 0.94 | 7.1 | 1258 | + |
| 55-end | 5 | 91 | 5.34 | 6.4 | ***** | − |

^{a}Incubation period is given as median values.
^{b}Percentage of infectivity is calculated from log⁻¹ of the ratio between treatment groups and the control (only for statistically significant differences).

Measurements of incubation time for disease, defined as the time from inoculation to the onset of clinical signs of neurological dysfunction, were transformed to titer values through established mathematical equations [Prusiner, S. B. et al Adv Exp Med Biol 134, 385-99 (1981)]. Further statistical analysis was performed by one way analysis of variance (Anova) and the non-parametric Kruskal-Wallis test, followed by a Comparison of all Groups vs. Control Group (Dunn's Method).

A statistically significant increase in the incubation time for the disease was found when comparing the control group with the groups in treatment plans 0-end or 14-end. In contrast, the 0-14 group showed a significant deleterious effect. For animals DMSO-treated from 55 dpi-end, no statistically significant difference from control group was observed.

Scrapie Infectious Rate in IP-Inoculated Animals Treated with DMSO

The intraperitoneal method of inoculation simulates better a peripheral infection since the pathogenic agent is not inoculated directly to the brain, where the active site of the disease is located. Instead, the inoculation occurs in the peritoneum, from where the infectious agent has to find its way to the brain.

Using this method does not allow the use of the ‘Incubation time assay’ method that was developed for calculation of infectious values in a logarithmic fashion, as it was possible for the results of IC-inoculated animals [Prusiner, S. B. et al (1981) ibid.]. Hence, infectious values here are expressed in days of disease incubation.

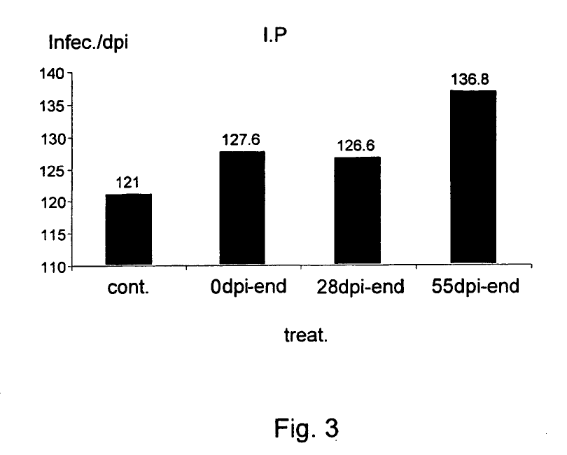

IP-infected animals were divided in four groups. Each one received DMSO according to one of the following treatment plans: control (no DMSO), from 0 dpi to disease (0-end), from 28 dpi to disease (28-end), or from 55 dpi to disease (55-end). These results are summarized in Table 2 and FIG. 3.

TABLE 2
Oral DMS0 treatment to IP Scrapie infected Hamsters
decreases infectious rate
Summary of the results of infectious rates in DMS0-treated
animals under different treatment plans comparing to control
untreated animals. The higher value indicates increased
animal life span, hence lower infectious rates.

| | | Incubation | | Variance from |
|---|---|---|---|---|
| DMS0 | Number of | period | | control group |
| treatment (dpi) | animals | (days) | Standard error | [P < 0.05]^{a} |
| control | 5 | 122 | 2.25 | |
| 0 - end | 5 | 127.6 | 0.98 | + |
| 28 - end | 5 | 126.6 | 1.94 | + |
| 55 - end | 5 | 136.8 | 3.69 | + |

^{a}[+] means statistically significant difference [p < 0.05]. All groups were tested as described for a Comparison vs. Control Group (Dunn's Method).

The results described in Table 2 indicate an overall beneficial effect for the animals treated with DMSO. An interesting finding is that animals treated only from 55 dpi onwards showed the most remarkable longevity in this model system. For the groups ‘0-end’and ‘28-end’, this effect was marginal. These results can be explained by the competition between anti-disease and adverse DMSO effects, such as weight loss. Since the IC model illustrated no significant effect for animals treated at relatively later stages of the disease, no treatments were started later than 55 dpi. Statistical analysis was done as described in the IC model. All groups showed a significant degree of variance [p<0.05] from control.

### Example 2

### DMSO Delays PrP^{Sc} Accumulation in the Brain

To compare the accumulation of PrP^{Sc} in the brain of DMSO-treated and untreated hamsters during disease incubation, we immunoblotted samples from brains collected from IC-inoculated animals. Since whole brain homogenates were used, differences in the distribution of PrP^{Sc} between different brain regions could not be addressed. Samples from untreated animals were compared to the DMSO-treated ‘14-end’ group. The samples were collected following euthanasia of the animals at 60, 90 and 110 dpi. The later time-point was only possible for DMSO-treated hamsters, since the maximum life span for untreated animals was 90 dpi.

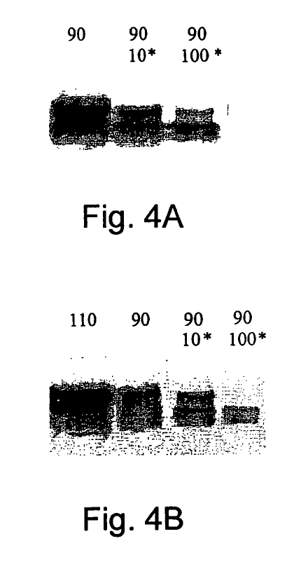

Interestingly, the samples from the untreated animals at final stages of the disease showed equivalent amounts of PrP^{Sc} as those from DMSO-treated animals at 110 dpi. Moreover, the samples from untreated animals at 90 dpi displayed bands of about 10 times higher intensity than those from DMSO-treated animals at 90 dpi (i.e. sacrificed simultaneously) (compare the bands from FIG. 4a with the bands from FIG. 4b).

This difference in amount of both PrP isoforms in the brain was already evident in samples from animals sacrificed at 60 dpi. At this stage, the ratio between treated/untreated samples was of 100 fold. This finding demonstrates that at the earlier time point the amount of PrP isoforms in an untreated Scrapie sick animal is equivalent to that of a DMSO-treated animal at 90-110 dpi (FIG. 5).

### Example 3

### Treatment of Terminally Ill Patients with DMSO

All the cases described in this section were patients that approached the Department of Neurology of the Hadassah Hospital in Jerusalem. These patients were diagnosed with CJD and underwent treatment with DMSO. The treatment comprised ingesting 25 ml of a 20% (w/v) aqueous solution of DMSO, three times per day. Daily urine tests of the patients treated with DMSO showed continuously increasing amounts of secreted PrP^{Sc} in the urine. This may be a result of DMSO preventing aggregation of PrP^{Sc} in the brain, thus facilitating its excretion.

Patient A:

46 years old, female, Jewish Libyan origin.
Family history: mother died after diagnosed with CJD.
First symptoms: sudden start of walk unsteadiness and vertigo.
Diagnosis: 4 months after the first symptoms the patient was hospitalized and a cerebellar syndrome, with pyramidal signs was found. The patient was originally diagnosed with an inflammatory demyelinating disease and treated with corticosteroids.

Six months after the first symptoms the patient was admitted in the hospital for a second time, after having developed cognitive decline. The patient was then diagnosed with CJD based on the following findings: presence of 14-3-3 protein in the cerebrospinal fluid, brain biopsy revealed positive histochemistry for PrP^{Sc}, and heterozygosity for a mutation in codon 200 of the PrP encoding gene.

Seven months after the first symptoms this patient started to be treated with DMSO. At this stage, the patient was restricted to a wheelchair due to her cerebellar syndrome and her cognition was severely impaired.

After five months of treatment, the patient was still alive, with an EEG showing slow activity but no actual periodicity. Medical evaluation determined that there was improvement in the patient's titubation and tremor, and that her deterioration was slower than expected for CJD patients, based on the fact that she still spoke a few words, and was able to swallow by herself and move her arms.

Patient B:

40 years old, male, Jewish Libyan origin.
Family history: uncles and aunts had died of a rapid progressive neurological illness.
First symptoms: headache and insomnia, followed within a month by vision disturbances, tremor, walking unsteadiness.
Diagnosis: CJD, based on the presence of 14-3-3 protein in the cerebrospinal fluid, and on the detection of homozygosity for a mutation in codon 200 of the PrP encoding gene.

DMSO treatment was started two months after the first symptoms. At this stage, the patient had only slight cognitive impairment, moderate cortical vision disturbance and severe cerebellar syndrome, but he was still able to walk with the help of a walker.

Six weeks after starting the treatment the patient died due to aspiration pneumonia.

Medical evaluation determined that during the treatment, there was no further cognitive decline, although the cerebellar syndrome progressed.

Patient C:

55 years old, male, Jewish Libyan origin.
Family history: not reported.
First symptoms: walking unsteadiness, dizziness and tinnitus.
Diagnosis: six months after the first symptoms the patient was diagnosed with CJD.

One year after the first symptoms the patient was hospitalized. At this stage the patient was bedridden, and presented no talking capacity and severe myoclonic jerks. The patient rapidly became comatose, due to aspiration pneumonia.

DMSO treatment was started through a gastric tube, but the patient died of sepsis three days after the beginning of the treatment, so the effect of DMSO on the disease could not be assessed.

1-25. (canceled)

26. A method comprising utilizing DMSO in the treatment and/or delay of the onset of clinical symptoms and/or delay of the progress of a neurodegenerative disorder caused by prions, said disorder being any one of CJD, vCJD, Kuru, GSS syndrome and FFI, specifically a Creutzfeldt-Jakob disease, wherein in said method said DMSO is administered orally.

27. A method comprising utilizing DMSO in preventing the agregation of PrP^{Sc} in the brain and/or facilitating the urinary excretion of PrP^{Sc} in patients suffering from a progressive neurodegenerative disorder caused by prions.

28. A method comprising utilizing DMSO in preventing the agregation of PrP^{Sc} in the brain and/or facilitating the urinary excretion of PrP^{Sc} in patients suffering from a progressive neurodegenerative disorder caused by prions, thereby alleviating or delaying the onset of said disorder.

29. The method of claim 27, wherein said neurodegenerative disorder is any one of Creutzfeldt-Jacob disease (CJD), variant CJD (vCJD), Kuru, Gerstmann-Sträussler-Scheinker syndrome (GSS) and fatal familial insomnia (FFI), specifically a Creutzfeldt-Jakob disease.

30. The method of claim 28, wherein said neurodegenerative disorder is any one of Creutzfeldt-Jacob disease (CJD), variant CJD (vCJD), Kuru, Gerstmann-Sträussler-Scheinker syndrome (GSS) and fatal familial insomnia (FFI), specifically a Creutzfeldt-Jakob disease.

31. A method for preparation of a pharmaceutical composition for the treatment and/or delaying the onset of clinical symptoms and/or delaying the progress of neurodegenerative disorder caused by prions, comprising utilizing as active ingredient DMSO, and optionally further comprising utilizing pharmaceutically acceptable additives and/or diluents, wherein said DMSO is administered orally.

32. The method of claim 31, wherein said neurodegenerative disorder is any one of Creutzfeldt-Jacob disease (CJD), variant CJD (vCJD), Kuru, Gerstmann-Sträussler-Scheinker syndrome (GSS) and fatal familial insomnia (FFI), specifically a Creutzfeldt-Jakob disease.

33. The method of claim 31, wherein said pharmaceutical composition is in the form of an aqueous solution of DMSO.

34. The method of claim 32, wherein said pharmaceutical composition is in the form of an aqueous solution of DMSO.

35. The method of claim 33, wherein the concentration of DMSO in said pharmaceutical composition is from about 1% (w/v) to about 100% (w/v).

36. The method of claim 35, wherein said concentration of DMSO is from about 5% (w/v) to about 50% (w/v).

37. The method of claim 36, wherein said concentration of DMSO is 20% (w/v).

38. The method of claim 31, wherein said pharmaceutical composition is for one to six times per day administration.

39. The method of claim 38, wherein said pharmaceutical composition is for two to four times per day administration.

40. The method of claim 39, wherein said pharmaceutical composition is for three times per day administration.

41. The method of claim 32, wherein the dose of DMSO per patient per day is about 0.2 g/kg.

42. The method of claim 32, wherein the dose of DMSO per patient per day is from about 0.1 g to about 30 g.

43. The method of claim 42, wherein the dose of DMSO per patient per day is from about 1 g to 15 g.

44. A method of treating and/or delaying the onset of clinical symptoms and/or delaying the progress of CJD, comprising orally administering to a CJD patient a therapeutically effective amount of DMSO or of a pharmaceutical composition as defined in claim 31.

45. The method of claim 44, comprising administering to said patient from about 3 g to about 30 g, preferably 15 g per day of DMSO.

46. A method of preventing the agregation of PrP^{Sc} in the brain and facilitating the urinary excretion of PrP^{Sc} in a patient suffering from a progressive neurodegenerative disorder caused by prions, comprising orally administering to said patient a therapeutically effective amount of DMSO or of a pharmaceutical composition as defined in claim 31.

47. A method of preventing the agregation of PrP^{Sc} in the brain and facilitating the urinary excretion of PrP^{Sc} in a patient suffering from a progressive neurodegenerative disorder caused by prions, comprising orally administering to said patient a therapeutically effective amount of DMSO or of a pharmaceutical composition as defined in claim 31, thereby alleviating or delaying the onset of said disorder.

48. The method of claim 46, wherein said neurodegenerative disorder is any one of Creutzfeldt-Jacob disease (CJD), variant CJD (vCJD), Kuru, Gerstmann-Sträussler-Scheinker syndrome (GSS) and fatal familial insomnia (FFI), specifically a Creutzfeldt-Jakob disease.

49. The method of claim 47, wherein said neurodegenerative disorder is any one of Creutzfeldt-Jacob disease (CJD), variant CJD (vCJD), Kuru, Gerstmann-Sträussler-Scheinker syndrome (GSS) and fatal familial insomnia (FFI), specifically a Creutzfeldt-Jakob disease.

50. The method of claim 46, wherein said administration is via the oral route.

51. The method of claim 47, wherein said administration is via the oral route.

52. The method of claim 46, comprising administering to said patient from about 3 g to about 30 g, preferably 15 g per day of DMSO.

53. The method of claim 47, comprising administering to said patient from about 3 g to about 30 g, preferably 15 g per day of DMSO.
